(19) United States
(12) Patent Application Publication
(10) Pub. No.: US 2006/0142772 A1
(43) Pub. Date: Jun. 29, 2006

(54) Surgical fasteners and related implant devices having bioabsorbable components

(76) Inventors: James D. Ralph, Bethlehem, PA (US); Stephen L. Tatar, Montville, NJ (US); Thomas N. Troxell, Pottstown, PA (US)

Correspondence Address: NORRIS MCLAUGHLIN & MARCUS, P.A., P O BOX 1018, SOMERVILLE, NJ, 08876, US

(21) Appl. No.: 11/025,213

(22) Filed: Dec. 29, 2004

(51) Int. Cl. A61B 17/58

(52) U.S. Cl. 606/76

(57) ABSTRACT

Surgical implants including fasteners and related devices which are partially bioabsorbable. The implants are constructed of a non-bioabsorbable base comprising metals and or high strength plastic materials. The base is partially or completely coated with a bioabsorbable material which can have its own mechanical features, such as the threads on a screw. Attachment elements are provided on the base to enhance the mechanical attachment of the bioabsorbable material to the base. The implants can withstand the torques and stresses encountered during surgery and, following implantation, in the body.

### BACKGROUND OF THE INVENTION

1. Field of the Invention

The present invention has to do with surgical fasteners and related devices which are implanted in the body. In particular, the invention has to do with improved surgical implants which are made with a base comprising metals and/or high strength plastic materials. Bioabsorbable materials are molded or otherwise applied onto the base and attached thereto. The base is made with elements which enhance the mechanical attachment of the bioabsorbable material to the base. The products of the invention can accordingly withstand the torques and stresses encountered during surgery and following surgery, in the body, while providing the benefits of bioabsorbability to the patient.

2. The Related Art

Bioabsorbable surgical fastening devices such as screws, pins, tacks, bolts, nails, suture anchors, staples, etc. and related devices such as rods, plates, wires and the like are now available for use in surgery. Such devices are commonly used in bone-to-bone, soft tissue-to-bone or soft tissue-to-soft tissue fixation. Previously, these devices were available only in metal or non-bioabsorbable high strength plastic materials.

Advances in the development of stronger bioabsorbable polymers have been made such as described in U.S. Pat. No. 6,406,498. However, for fastening applications and other applications which require strong materials, bioabsorbable substances usually do not have sufficient strength to withstand the stresses encountered during surgery and, following surgery, in the body. When such materials fail during surgery, more extensive and prolonged surgery may be required. And when such materials fail in the body, re-surgery is usually required.

Coated implants have been described to a limited extent and for limited purposes such as in U.S. Pat. No. 5,571,139 which describes a bidirectional suture anchor which can be constructed of a non-absorbable biocompatible material coated with a bioabsorbable coating having a low coefficient of friction. The low coefficient of friction is said to assist in the installation of the implant.

The present invention overcomes problems associated with the prior art by providing surgical fasteners and related devices with bioabsorbable components which are sufficiently attached to an underlying, non-bioabsorbable base that they will not separate from the base during surgery.

In some embodiments the present invention also provides implants having a base coated with bioabsorbable material which itself has functional mechanical features, such as bioabsorbable threads, which are not embodied in the base.

### SUMMARY OF THE INVENTION

The invention has to do with improved surgical fasteners and related devices having as a base a tissue friendly metallic material or high strength plastic material which is not bioabsorbable. The fasteners include any kind of surgical fastener which may be completely or partially implanted in the body. Examples of such fasteners include screws, cannulated screws, suture anchors, bone achors, pins, tacks, bolts, nails, staples, etc. Related devices include rods, plates, wires and the like. The base is completely or partially coated with a bioabsorbable material and the bioabsorbable material is attached to the base.

The base is made with attachment elements which provide for the mechanical attachment of the bioabsorbable material thereto. This can include, for example, indentations, protrusions, corrugations, partial or through holes, slits, nubs, pockets, bumps, splines, knurls; mechanical roughening such as by sanding, sand blasting, bead blasting, shot peening, tumbling or etching; coating or plating with an adherency enhancing alloy or other composition or the like. Combinations of two or more of such attachment elements can also be employed. Alternatively, the base can be made from a mesh material or cable which provides for attachment of the bioabsorbable material by means of the structure of the mesh or cable. The objective in selecting a material for the base, in addition to biocompatibility, is to find a material having sufficient strength for the particular application but also, in many cases, sufficient flexibility when implanted in the body. For example, a screw that is sufficiently flexible to allow micro-motion will be less likely to back out, will absorb shock and may generate or stimulate bone growth.

The bioabsorbable material optionally can have functional mechanical features which are not features of the base. For example, when the fastener is a screw, the bioabsorbable material can be threaded even though the base is not threaded.

### BRIEF DESCRIPTION OF THE DRAWINGS

The drawings are intended to be illustrative, are not drawn to scale and are not intended to limit the scope of the claims to the embodiments depicted.

FIGS. 1-4 are views of various surgical screws according to the invention.

FIG. 5 is a section view of a base for a surgical screw of the invention.

FIGS. 6-17 are views of various base designs for surgical fasteners of the invention.

FIG. 18 is a section view of a base for a surgical screw of the invention.

FIG. 19 illustrates a surgical screw of the invention.

FIG. 20 is a section view of a base for a surgical screw of the invention.

FIG. 21 is a section view of a base for a surgical screw of the invention.

FIGS. 22 and 23 illustrate two pin designs according to the invention.

FIG. 24 illustrates a tack according to the invention.

FIG. 25 illustrates a bolt according to the invention.

FIG. 26 illustrates a nail according to the invention.

FIG. 27 illustrates a staple according to the invention.

FIG. 28 illustrates a rod according to the invention.

FIG. 29 illustrates a wire according to the invention.

FIG. 30 illustrates a plate according to the invention.

FIGS. 31 and 32 illustrate a screw and washer combination according to the invention.

FIG. 33 illustrates six different head configurations for the screws of the invention and one head configuration for the pins of the invention.

It should be noted that many of the illustrated bases for screws can also be used for pins and the various illustrations of attachment elements can be used on any if not all of the fasteners and related devices of the invention as will be apparent to those skilled in the art.

### DESCRIPTION OF THE PREFERRED EMBODIMENTS

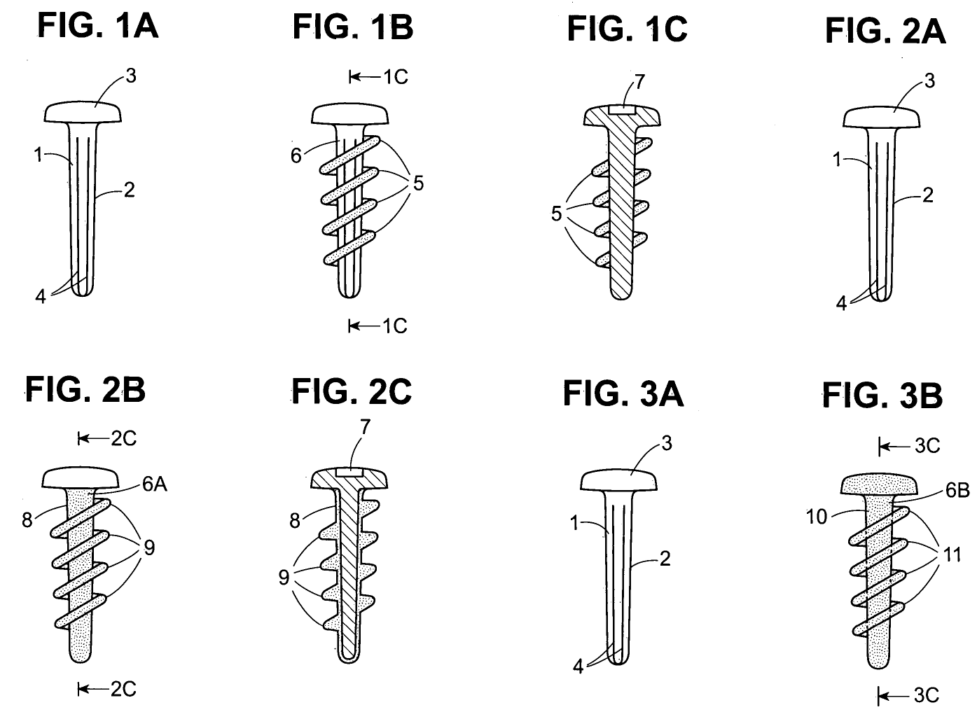

FIG. 1A illustrates a base 1 having a shank 2 and a head 3. The shank 2 contains grooves 4 which serve as attachment elements. In FIG. 1B bioabsorbable threads 5 have been attached to the base to make screw 6. FIG. 1C is a section view of screw 6 taken along section line a-a of FIG. 1B. Slot 7 is illustrated in the head. Following implantation of screw 6 and the eventual absorption by the body of threads 5, the base 1 can optionally be removed smoothly from the body. In the prior art, when non-bioabsorbable screws are used, the threads may stick to the surrounding tissue and in such cases removal of the screws can cause injury to the patient. This problem is avoided when the screws of the present invention are used.

FIG. 2A illustrates the same base as illustrated in FIG. 1A. In FIG. 2B a bioabsorbable coating 8 with bioabsorbable threads 9 have been coated over and attached to the shank of the base to make screw 6A. This embodiment is illustrated further in FIG. 2C which is a section taken along section line a-a of FIG. 2B. The bioabsorbable coating 8 and bioabsorbable threads 9 are easily identified in section.

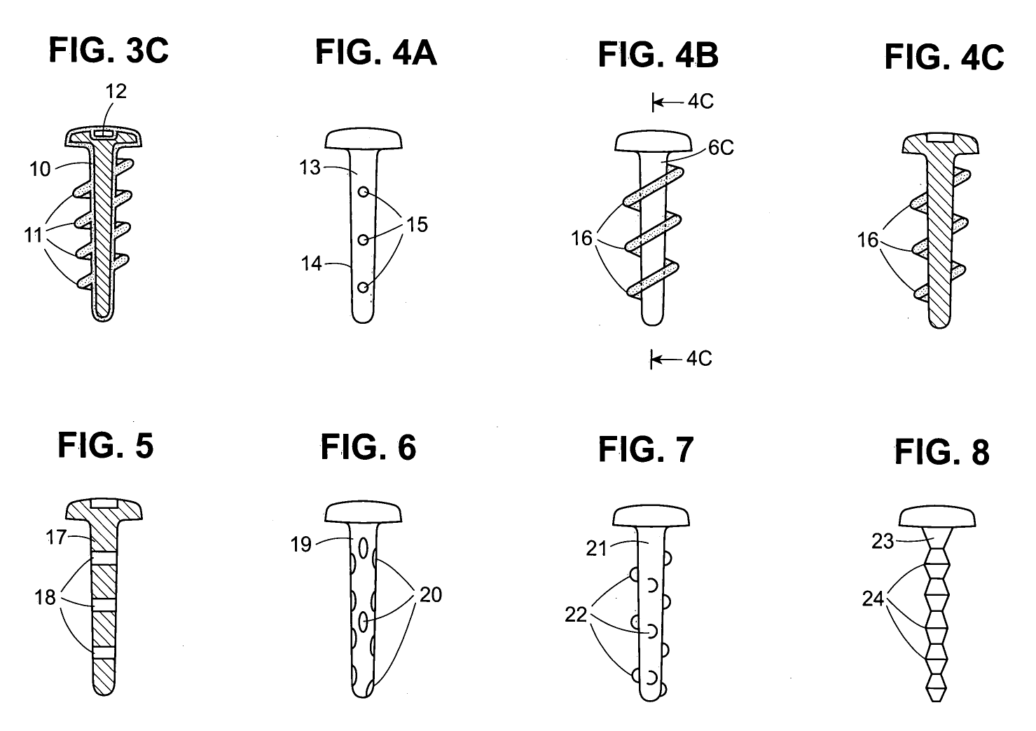

FIG. 3A also illustrates the same base as illustrated in FIG. 1A. FIG. 3B illustrates screw 6B having bioabsorbable coating 10 and bioabsorbable threads 11 coated over and attached to the base 1. The section illustration of FIG. 3C is taken along section line a-a of FIG. 3B and illustrates base 1 entirely coated with bioabsorbable material including coated slot 12.

FIG. 4A illustrates a base 13 having a shank 14 with partial holes 15 therein. Partial holes 15 are the attachment elements. FIG. 4B illustrates a screw 6C made by adhering bioabsorbable threads 16 onto the base 13. FIG. 4C illustrates screw 6C in section with the section being taken along section line a-a of FIG. 4B.

FIG. 5 illustrates in section a base 17 for a surgical screw. Through holes 18 are the attachment elements.

FIG. 6 illustrates base a 19 for a surgical screw having indentations or pockets 20 as the attachment elements.

FIG. 7 illustrates base 21 for a surgical screw having protrusions, bumps or nubs 22 as the attachment elements.

FIG. 8 illustrates base 23 for a surgical screw having corrugations 24 as the attachment elements.

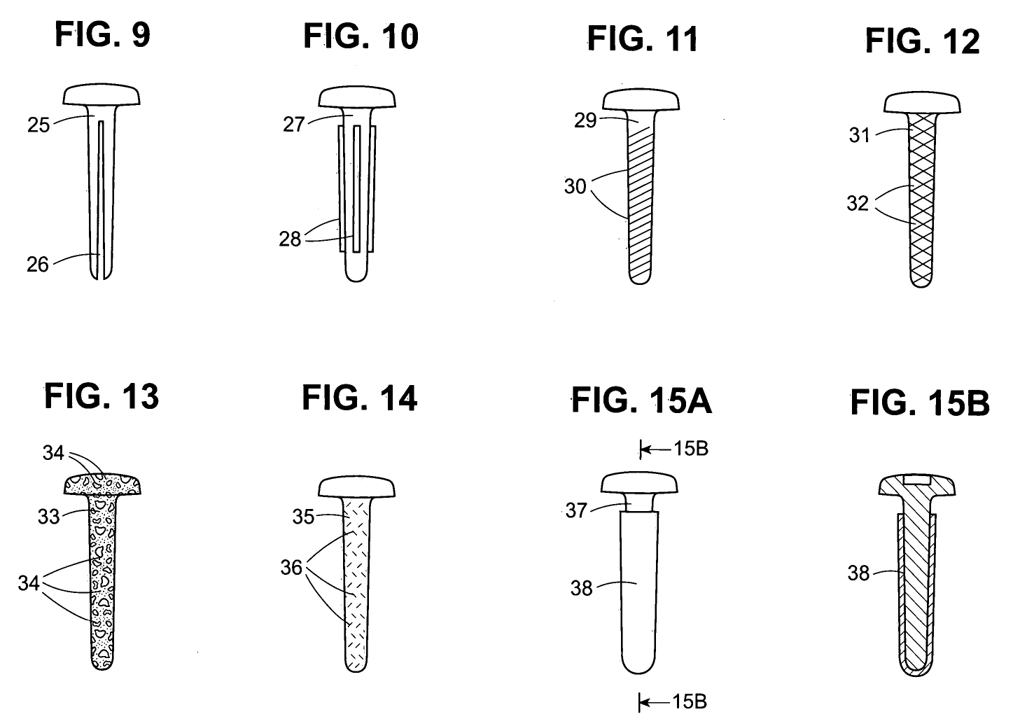

FIG. 9 illustrates a base 25 for a surgical screw having a through slit 26 as the attachment element.

FIG. 10 illustrates a base 27 for a surgical screw having splines 28 as the attachment elements.

FIG. 11 illustrates a base 29 for a surgical screw having threads 30 as the attachment elements.

FIG. 12 illustrates a base 31 for a surgical screw having knurls 32 as the attachment elements.

FIG. 13 illustrates a base 33 for a surgical screw having a roughened surface 34 as the attachment elements. The roughened surface can be imparted to the base by, for example, sanding or sand blasting. It should be noted that the attachment elements 34 are illustrated over the entire surface of base 33 and this design is suitable for screws, pins and other implant devices that will be completely coated with the bioabsorbable material. Other attachment elements can be completely or partially applied to any base of the invention and combinations of two or more than two attachment elements can be used as will be apparent to those skilled in the art.

FIG. 14 illustrates a base 35 having an etched surface 36 as the attachment elements.

FIG. 15A illustrates a base 37 for a surgical screw having an adherency promoting surface coated or plated thereon. The adherency promoting surface can also be seen in FIG. 15B which is a section view of FIG. 15A taken along section line a-a.

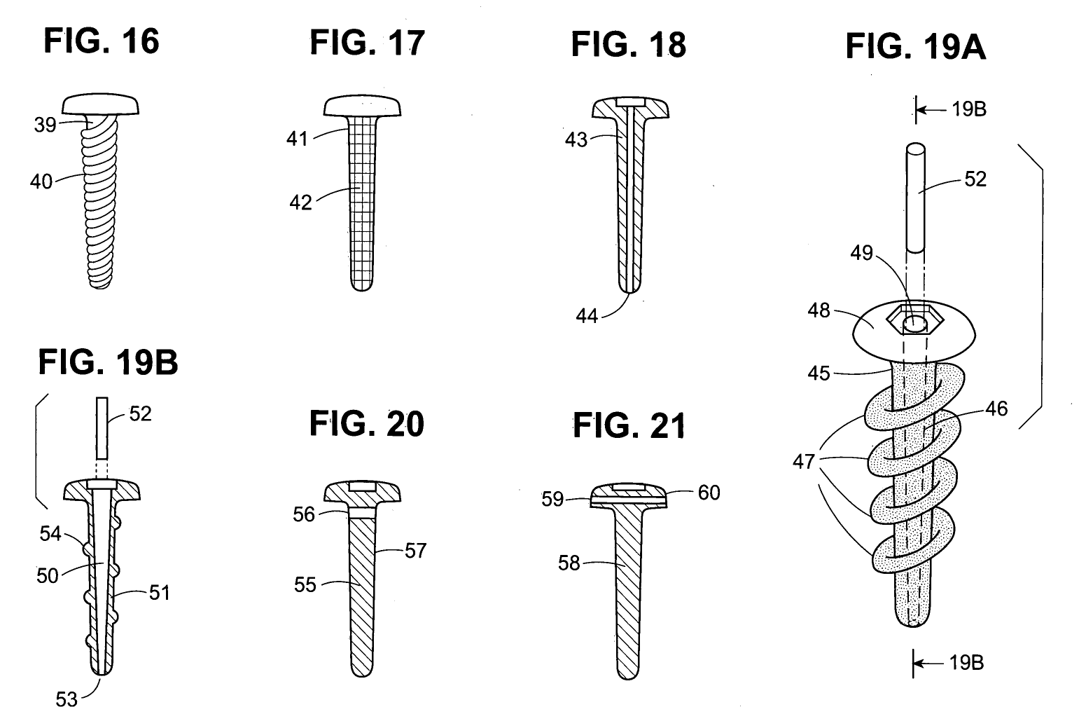

FIG. 16 illustrates a base 39 for a surgical screw having a shank 40 comprised of a cable.

FIG. 17 illustrates a base 41 for a surgical screw having a shank 42 comprised of or covered with a mesh material.

FIG. 18 illustrates in section a base 43 for a surgical screw having a bore 44 through the entire longitudinal length thereof. This base can be used to make a cannulated screw or pin according to the invention.

FIG. 19A illustrates a surgical screw 45 having a shank coated with a bioabsorbable coating 46 and bioabsorbable threads 47. The head 48 is illustrated in top view FIG. 19Aa. Opening 49 is in open communication with a tapered slit 50 which is illustrated in FIG. 19B. FIG. 19B illustrates the base for the screw 45 before it was coated with the bioabsorbable material. Pin 52 illustrated in FIG. 19A can be inserted into opening 49 and driven into the slit 50. The diameter of pin 52 is less than the diameter of the opening 49 and greater than the width of the slit at distal end 53. After the screw 45 is implanted in a patient, the pin 52 can be driven into the screw to make a tighter fit, recognizing that the bioabsorbable coating 46 and threads 47 may have to expand or crack but the fixation of the screw in the patient is nevertheless improved. It is noted that nubs 54 are optionally provided as attachment elements and the slit 50 also serves as an attachment element.

FIG. 20 illustrates in section a base 55 for a surgical screw of the invention. Transverse bore 56 is made across and through the entire diameter of the shank 57.

FIG. 21 illustrates in section base 58 for a surgical screw of the invention. Transverse bore 59 passes across and through the entire diameter of head 60.

It is noted that the bore 44 in FIG. 18, bore 56 in FIG. 20 and bore 59 in FIG. 21 can accommodate sutures and the screws made according to the invention with the bases illustrated in those drawings can be used as suture anchors or bone anchors.

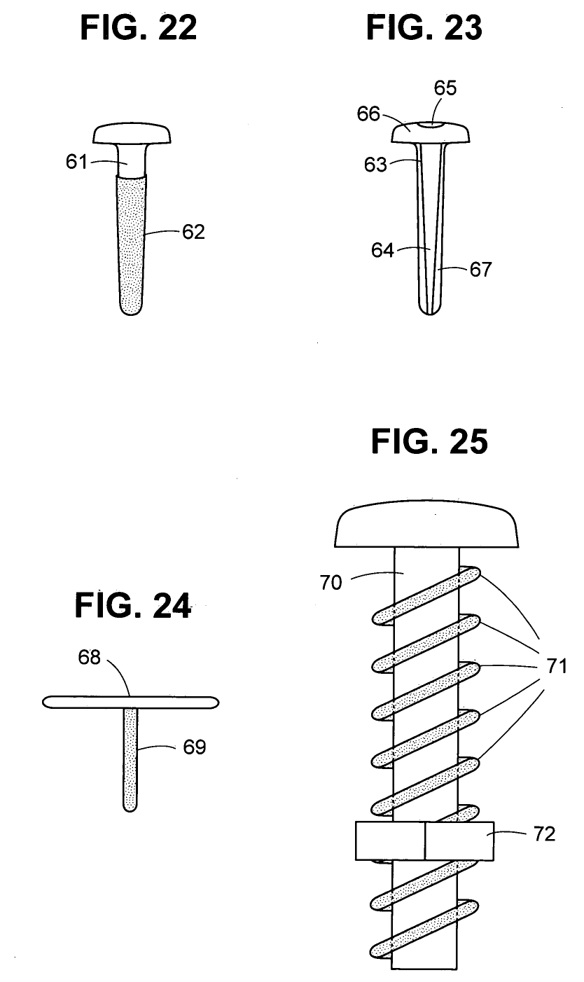

FIG. 22 illustrates a pin 61 of the invention having bioabsorbable coating 62 thereon.

FIG. 23 illustrates a pin 63 of the invention having a tapered slit 64 which communicates with opening 65 in head 66. The pin is coated with bioabsorbable coating 67. A pin 52 of the type illustrated in FIG. 19A can be driven into pin 63 to fix the pin 63 in position in the patient.

FIG. 24 illustrates a tack 68 having a partial bioabsorbable coating 69 thereon and attached thereto.

FIG. 25 illustrates a bolt 70 having bioabsorbable threads 71 attached thereto and a nut 72.

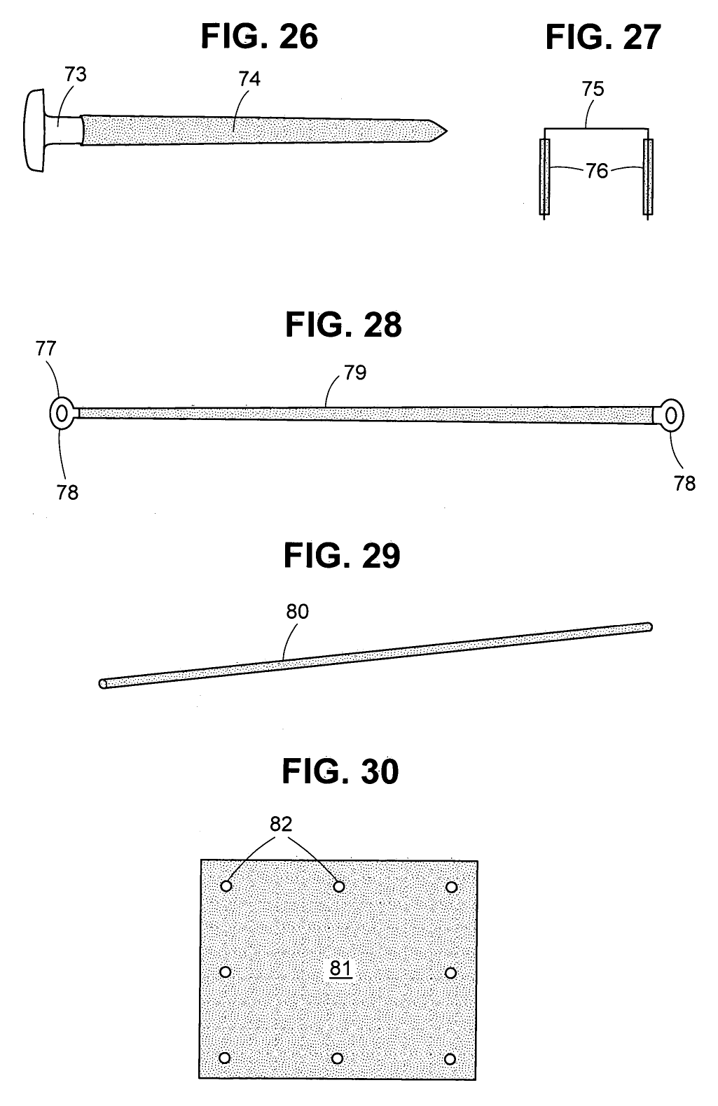

FIG. 26 illustrates a nail 73 having a bioabsorbable coating 74 thereon and attached thereto.

FIG. 27 illustrates a staple 75 having bioabsorbable coatings 76 thereon and attached thereto.

FIG. 28 illustrates a rod 77 having mounting holes 78 and a bioabsorbable coating 79 thereon and attached thereto.

FIG. 29 illustrates a wire 80 which has a bioabsorbable coating over the surface thereof and attached thereto.

FIG. 30 illustrates a plate 81 having mounting holes 82. A bioabsorbable coating is provided over the surface thereof and attached thereto.

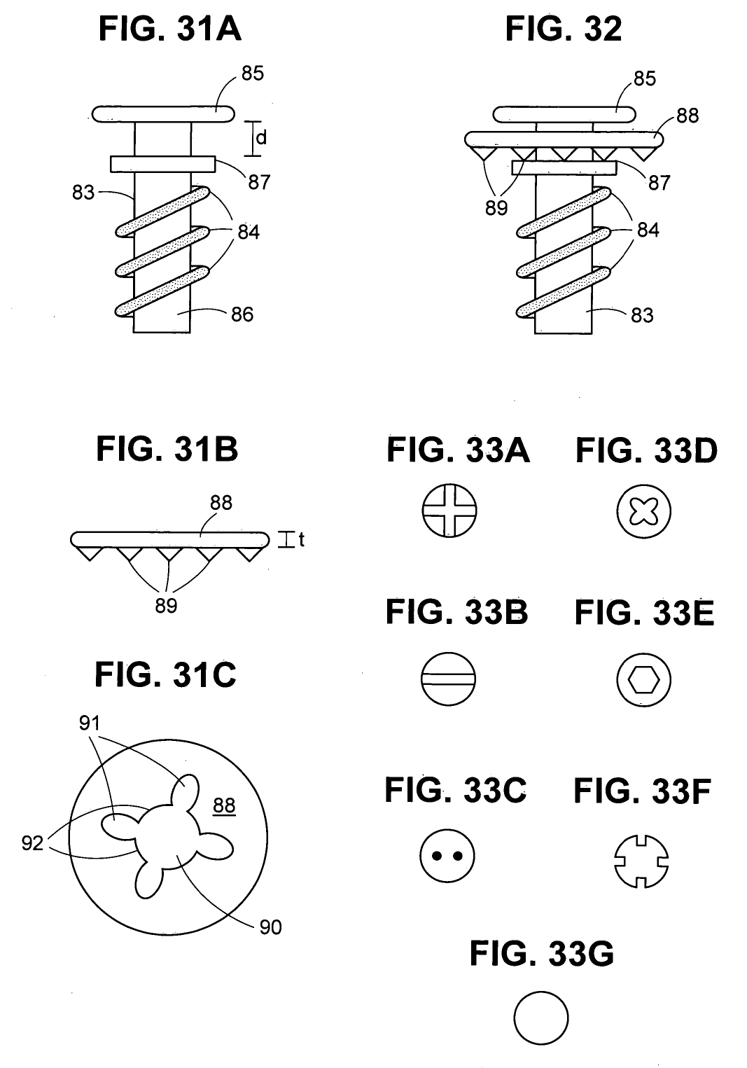

FIG. 31A illustrates screw 83 having bioabsorbable threads 84. The screw 83 has a head 85 affixed to shank 86. Annular ring 87 is affixed to shank 86. Washer 88 illustrated in FIGS. 31B and C is provided with protuberances 89, center opening 90, radial openings 91 and fingers 92. The fingers are somewhat flexible and permit the washer to snap over ring 87 so that the washer will stay rotatably disposed between the head 85 and ring 87 as illustrated in FIG. 32. The distance d between the head 85 and the ring 87 is greater than the thickness t of washer 88 and the radius of center opening 90 is greater than the outer radius of the threads 84 but is less than the outer radius of ring 87. When the screw is installed at the operating site in a patient, protuberances 89 contact and enter the operating surface and prevent the washer from rotating as the screw is tightened down. The combination of screw 83 and washer 88 is particularly suitable as a tissue anchor.

FIG. 33 illustrates top views of various head configurations for the screws and pins of the invention. FIG. 17 A is a Philips head, B is a slotted head, C is a spannon, D is a torq, E is a hex and F is a new design. Head G can be used for a pin.

Base designs of the invention have been illustrated for various types of screws and pins but, as will be apparent to those skilled in the art, the base can have various shapes and sizes depending on the design requirements of the end product. One skilled in the art can design any implant according to the invention bearing in mind the limitations and advantages of the materials used. A suitable base can be designed accordingly for any end product that will be completely or partially coated with a bioabsorbable material, bearing in mind that the bioabsorbable material itself can have mechanical features such as protrusions, indentations, threads and the like.

Suitable materials for the base of the invention include tissue friendly metals, alloys, synthetic metals, ceramics, plastics and reinforced plastics which are commonly used in surgical implants of all kinds. Such materials include materials that have sufficient strength to meet the objectives of the invention and that have been approved by the United States Food and Drug Administration (FDA) for surgical implant applications.

Generally speaking, there are three main types of alloys used in orthopedic metals today, titanium alloys, cobalt alloys and iron alloys. An exhaustive list is available on the FDA website which also provides the reference numbers and effective dates of the ASTM or ISO standards for the materials. Some examples include unalloyed and alloyed titanium; molybdenum, chromium, cobalt, tungsten, aluminum, niobium, manganese or vanadium in various combinations as alloys or components of alloys, various stainless steels and other iron alloys; aluminum oxides, zirconium oxides, tantalum and calcium phosphates.

Numerous types of high strength plastic materials that are not considered to be bioabsorbable also are employed to make implants and many of these are identified not only on the FDA website mentioned above but also on the ASTM website. Examples of suitable high strength plastic materials include polyetheretherketone (PEEK), epoxys, polyurethanes, polyesters, polyethylenes, vinyl chlorides, polysulfones, polytetrafluoro-ethylene (PTFE), polycarbonates, polyaryletherketone (PAEK), polyoxymethylene, nylon, carbon fiber polyester, polyetherketoneetherketoneketone (PEKEKK), silicones and the like. When a plastic material is used, a small wire or other material can be incorporated in the main body of the base for purposes of x-ray detection.

The foregoing lists of materials may have application in some embodiments of the present invention but not in others as will be apparent to those skilled in the art based on requirements of strength, flexibility, machinability and the like for the particular application. The lists are intended to be illustrative and not exhaustive. Other materials and new materials may be employed based upon the principles of the invention as set forth herein.

For purposes of this specification, the term “high strength plastic material” is defined as any tissue-friendly non-bioabsorbable polymer, copolymer, polymer mixture or polymer alloy having sufficient strength to withstand without failure the torques and stresses that a fastener or related implant device of the invention would normally be subjected to during surgery or in the body.

The term “bioabsorbable material” as used herein includes materials which are partially or completely bioabsorbable in the body.

Suitable bioabsorbable materials include polyglycolide, poly(lactic acid), copolymers of lactic acid and glycolic acid, poly-L-lactide, poly-L-lactate; crystalline plastics such as those disclosed in U.S. Pat. No. 6,632,503 which is incorporated herein by reference; bioabsorbable polymers, copolymers or polymer alloys that are self-reinforced and contain ceramic particles or reinforcement fibers such as those described in U.S. Pat. No. 6,406,498 which is incorporated herein by reference; bioresorbable polymers and blends thereof such as described in U.S. Pat. No. 6,583,232 which is incorporated herein by reference; copolymers of polyethylene glycol and polybutylene terephthalate; and the like. The foregoing list is not intended to be exhaustive. Other bioabsorbable materials can be used based upon the principles of the invention as set forth herein.

Bioactive materials can be admixed with the bioabsorbable materials, impregnated in the bioabsorbable materials and/or coated on the outer surface thereof and/or coated on the base or otherwise provided at the interface of the base with the bioabsorbable material. These materials can include, for example, bioactive ceramic particles, capsules or reinforcement fibers and they can contain, for example, antimicrobial fatty acids and related coating materials such as those described in Published U.S. Patent Application No. 2004/0153125 A1; antibiotics and antibacterial compositions; immunostimulating agents; tissue or bone growth enhancers and other active ingredients and pharmaceutical materials known in the art.

The products of the invention can be made by molding, heat shrinking or coating the bioabsorbable material on a base which has been provided with attachment means. When the bioabsorbable material will have functional mechanical properties which are not made from the base material, the bioabsorbable material can be molded onto the base in the desired shape. Alternatively, the bioabsorbable material also can be coated, shrink wrapped or molded onto the base and then machined to the desired shape and/or dimensions.

What is claimed is:

1. A surgical fastener comprising a base, all or a portion of said base having attachment elements thereon and a bioabsorbable material overlaying all or a portion of said base and attached thereto, said base comprising a metal or a high strength plastic material, wherein the surgical fastener is selected from the group consisting of screws, cannulated screws, suture anchors, bone anchors, pins, tacks, bolts, nails and staples.

2. The fastener of claim 1 wherein the attachment elements are indentations, protrusions, corrugations, partial holes, through holes, slits, nubs, pockets, bumps, splines or knurls or a combination of two or more than two thereof.

3. The fastener of claim 1 wherein the attachment elements comprise a roughened or etched surface or a coated or plated adherency enhancing alloy or a combination of two or more than two thereof.

4. The fastener of claim 2 wherein the attachment elements further comprise a roughened or etched surface or a coated or plated adherency enhancing alloy or a combination of two or more than two thereof.

5. The fastener of claim 1 wherein the base comprises a mesh material or a cable.

6. The fastener of claim 1 selected from the group consisting of screws, cannulated screws, suture anchors and bone anchors having threads wherein the bioabsorbable material is threaded.

7. The fastener of claim 1 further comprising a bioactive material coated thereon.

8. A surgical implant device selected from the group consisting of rods, plates and wires comprising a base, all or a portion of said base having attachment elements thereon and a bioabsorbable material overlaying all or a portion of said base and attached thereto, said base comprising a metal or a high strength plastic material.

9. The surgical implant device of claim 8 wherein the attachment elements are indentations, protrusions, corrugations, partial holes, through holes, slits, nubs, pockets, bumps, splines or knurls or a combination of two or more than two thereof.

10. The surgical implant device of claim 8 wherein the attachment elements comprise a roughened or etched surface or a coated or plated adherency enhancing alloy or a combination of two or more than two thereof.

11. The surgical implant device of claim 9 wherein the attachment elements further comprise a roughened or etched surface or a coated or plated adherency enhancing alloy or a combination of two or more than two thereof.

12. The surgical implant device of claim 8 wherein the base comprises a mesh material or a cable.

13. The surgical implant device of claim 8 selected from the group consisting of screws, cannulated screws, suture anchors and bone anchors having threads wherein the bioabsorbable material is threaded.

14. The surgical implant device of claim 8 further comprising a bioactive material coated thereon.

15. A surgical screw comprising
a base having an elongated shank, all or a portion of said base having attachment elements thereon, and a head comprising a proximal surface that is substantially perpendicular to the longitudinal axis of the shank, said shank protruding distally from said head to a distal end, said head comprising one or more than one recess in said proximal surface, said base comprising a metal or a high strength plastic material, and
a bioabsorbable material overlaying all or a portion of said base and attached thereto and at least a portion of the outer surface of said bioabsorbable material is threaded.

16. The surgical screw of claim 15 having a bore extending through the entire length thereof from the proximal surface to the distal end.

17. The surgical screw of claim 15 wherein the head has a first diameter, and the shank has a second diameter less than the first diameter, and having a bore extending through the head the entire length of the first diameter or extending through the shank the entire length of the second diameter, said bore being substantially perpendicular to the longitudinal axis of the shank.

18. The surgical screw of claim 16 wherein the shank has an outer surface, the base has an inner surface and the bore is tapered so that the diameter of the bore at the proximal surface is greater than the diameter of the bore at the distal end and the shank has at least one longitudinal slit from the distal end toward the head and intersecting the outer surface of the shank and the inner surface of the bore.

19. The surgical screw of claim 18 wherein the bioabsorbable material has an outer surface and the at least one longitudinal slit also intersects said outer surface of the bioabsorbable material.

20. The surgical screw of claim 18 further comprising a cylindrical pin having a diameter less than the diameter of the bore at the proximal surface and greater than the diameter of the bore at the distal end wherein the pin is inserted in the bore.

21. The surgical screw of claim 19 further comprising a cylindrical pin having a diameter less than the diameter of the bore at the proximal surface and greater than the diameter of the bore at the distal end wherein the pin is inserted in the bore.

22. A surgical fastener comprising
a partially bioabsorbable screw comprising a base having an elongated shank, all or a portion of said base having attachment elements thereon, and a head having a first outer diameter comprising a proximal surface that is substantially perpendicular to the longitudinal axis of the shank, said shank protruding distally from said head to a distal end, said head comprising one or more than one recess in said proximal surface, said base comprising a metal or a high strength plastic material, and a bioabsorbable material overlaying all or a portion of said base and attached thereto, at least a portion of the outer surface of said bioabsorbable material being threaded and having an outer thread diameter;
an annular ring having a second outer diameter affixed circumferentially around the shank in proximity to the head but spaced from the head a distance along the shank, said second outer diameter being less than the first outer diameter and greater than the outer thread diameter;
a washer removably connected to said partially bioabsorbable screw and having a thickness and an opening of a first inner diameter, said thickness being less than the distance along the shank and said first inner diameter being greater than the outer thread diameter and less than the second outer diameter, fingers circumferentially spaced around said opening and having sufficient flexibility to permit the washer to be snapped over the annular ring, the washer having an upper surface facing the head and a lower surface having a plurality of protuberances.

23. The surgical fastener of claim 22 wherein the washer is bioabsorbable or partially bioabsorbable.

24. The surgical fastener of claim 22 wherein the washer is rotatably disposed between the head and the annular ring.

25. The surgical fastener of claim 22 further comprising a bioactive material coated thereon.

26. A surgical pin comprising
a base having an elongated shank, all or a portion of said base having attachment elements thereon, and a head comprising a proximal surface that is substantially perpendicular to the longitudinal axis of the shank, said shank protruding distally from said head to a distal end, said base comprising a metal or a high strength plastic material, and
a bioabsorbable material overlaying all or a portion of said base and attached thereto.

27. The surgical pin of claim 26 having a bore extending through the entire length thereof from the proximal surface to the distal end.

28. The surgical pin of claim 27 wherein the shank has an outer surface, the base has an inner surface and the bore is tapered so that the diameter of the bore at the proximal surface is greater than the diameter of the bore at the distal end and the shank has at least one longitudinal slit from the distal end toward the head and intersecting the outer surface of the shank and the inner surface of the bore.

29. The surgical pin of claim 28 wherein the bioabsorbable material has an outer surface and the at least one longitudinal slit also intersects said outer surface of the bioabsorbable material.

30. The surgical pin of claim 28 further comprising a cylindrical pin having a diameter less than the diameter of the bore at the proximal surface and greater than the diameter of the bore at the distal end wherein the pin is inserted in the bore.

31. A method of attaching soft tissue to a bone comprising
providing a surgical fastener comprising
a partially bioabsorbable screw comprising a base having an elongated shank, all or a portion of said base having attachment elements thereon, and a head having a first outer diameter comprising a proximal surface that is substantially perpendicular to the longitudinal axis of the shank, said shank protruding distally from said head to a distal end, said head comprising one or more than one recess in said proximal surface, said base comprising a metal or a high strength plastic material, and a bioabsorbable material overlaying all or a portion of said base and attached thereto, at least a portion of the outer surface of said bioabsorbable material being threaded and having an outer thread diameter;
an annular ring having a second outer diameter affixed circumferentially around the shank in proximity to the head but spaced from the head a distance along the shank, said second outer diameter being less than the first outer diameter and greater than the outer thread diameter;
a washer rotatably disposed between the head and the annular ring and having a thickness and an opening of a first inner diameter, said thickness being less than the distance along the shank and said first inner diameter being greater than the outer thread diameter and less than the second outer diameter, fingers circumferentially spaced around said opening and having sufficient flexibility to permit the washer to be snapped over the annular ring, the washer having an upper surface facing the head and a lower surface having a plurality of protuberances; and
inserting the distal end of the fastener through soft tissue and then inserting the distal end of the fastener into the bone.
